(19) United States
(12) Patent Application Publication
(10) Pub. No.: US 2017/0333049 A1
(43) Pub. Date: Nov. 23, 2017

(54) PYLORIC OBSTRUCTION DEVICE WITH SPACE-OCCUPYING DEVICE

(71) Applicant: EasyNotes Ltd., Kfar Truman (IL)

(72) Inventors: Izhak Fabian, Kfar Truman (IL); Moshe Mizrahy, Tel Aviv (IL)

(73) Assignee: EasyNotes Ltd., Kfar Truman (IL)

(21) Appl. No.: 15/600,904

(22) Filed: May 22, 2017

Related U.S. Application Data
(60) Provisional application No. 62/339,963, filed on May 23, 2016.

(51) Int. Cl. A61B 17/12; A61F 5/00

(52) U.S. Cl. CPC A61B 17/12136; A61F 5/0079; A61F 5/0036; A61B 17/12172; A61B 17/12036; A61B 17/12045; A61B 17/12099; A61B 17/12022; A61B 17/1219; A61F 5/003

(57) ABSTRACT

An assembly includes an obstruction device including a proximal obstruction balloon and a distal obstruction balloon mounted on a shaft, the balloons being inflatable via one or more inflation lumens, and a space-occupying device mounted on a positioning member that extends from a portion of the obstruction device, the space-occupying device being proximal to the proximal obstruction balloon and inflatable via a space-occupying-device inflation lumen.

### FIELD OF THE INVENTION

The present invention generally relates to devices for obstructing or reducing flow through a body lumen, in particular for obstructing or reducing flow of gastric contents across the pyloric valve combined with a space-occupying device in the stomach.

### BACKGROUND OF THE INVENTION

There have been many attempts in the past to surgically modify the gastrointestinal tract to reduce the desire to eat. Stomach saplings, or gastroplasties, to reduce the volumetric size of the stomach, therein achieving faster satiety, were performed in the 1980's and early 1990's, but were not very successful. The reasons are not all known, but are believed related to several factors. One of which is that the stomach stretches over time increasing volume while psychological drivers motivate patients to find creative approaches to literally eat around the smaller pouch.

Some of the currently used techniques in the prior art include the Roux-en-Y gastric bypass and the biliopancreatic diversion with duodenal switch (BPD). Both procedures reduce the size of the stomach plus shorten the effective-length of intestine available for nutrient absorption. Reduction of the stomach size reduces stomach capacity and the ability of the patient to take in food. Bypassing the duodenum makes it more difficult to digest fats, high sugar and carbohydrate rich foods. One objective of the surgery is to provide feedback to the patient by producing a dumping syndrome if they do eat these food products. Dumping occurs when carbohydrates directly enter the jejunum without being first conditioned in the duodenum. The result is that a large quantity of fluid is discharged into the food from the intestinal lining. The total effect makes the patient feel light-headed and results in severe diarrhea. For reasons that have not been determined the procedure also has an immediate therapeutic effect on diabetes.

Endoscopic procedures that have been used to assist weight loss have been primarily focused on the placement of a balloon or other space occupying device in the patient's stomach to fill portions of the stomach to provide the patient with the feeling of fullness, thereby reducing food intake. To accomplish these procedures, an endoscope is utilized to guide the balloon through the patient's mouth and down the esophagus to the stomach. Usually these procedures have allowed placement of the device for 6-12 months, and are coupled with counseling and other types of psychological support.

### SUMMARY OF THE INVENTION

The present invention seeks to provide an obstruction assembly for obstructing or reducing flow through a body lumen, in particular for obstructing or reducing flow of gastric contents across the pyloric valve (pylorus) combined with a space-occupying device, as is described more in detail hereinbelow. The assembly is particularly useful in a transoral gastrointestinal procedure, but the invention is not limited to transoral gastroplasty, and may be used in other laparoscopic, endoscopic, or natural orifice procedures in other body lumens. The pyloric plug (proximal and distal balloons) and the space-occupying device are designed to be fully operative over a long time, such as but not limited to, between six months and many years. The assembly can be removed, if desired, and can also be re-implanted.

The plug includes two balloons, one proximal and the other distal, mounted on a shaft. The proximal obstruction balloon is arranged to fit in the stomach, whereas the distal obstruction balloon is arranged to fit in the duodenum. When inflated, both balloons expand towards the pylorus from opposite sides on the pylorus, thus fixing the plug in place. The balloons may have any shape, such as but not limited to, spherical, cylindrical, T-shape with or without wings, polygonal, irregular and many others.

The space-occupying device may be a balloon that is mounted on a shaft that extends from the proximal balloon towards the stomach. In particular, the shaft is designed to position the space-occupying device at an effective place in the stomach, such as the upper stomach (fundus).

The plug is particularly useful to stop the flow of stomach contents to the proximal gut which includes the duodenum and the initial part of the jejunum. Such a need arises, for example, after creating an alternative path of flow through a gastro-jejunum anastomosis which bypasses the proximal gut. There could be other cases when this need arises, such as after surgery in the duodenum area or in the pancreas or bile outputs to the duodenum. Another indication could be the need to operate endoscopically on the stomach with an inflated stomach. In this case, the plug keeps the inflating air in the stomach and it does not bloat the intestine.

The plug can be used in a method for creating an anastomosis between a stomach and a portion of a small intestine, wherein the plug is used to control passage of stomach contents through the pylorus during and after creation of the anastomosis. For example, before the anastomosis has been created, the plug would allow passage of material therethrough, but after creation of the anastomosis the pylorus plug would either completely block flow (so that material only flows through the anastomosis) or partially block flow (so that material can flow through both the plug and the anastomosis).

A delivery system may be used to insert the obstruction assembly, retrieve it, and inflate and deflate the proximal and distal balloons and the space-occupying device of the assembly.

There is thus provided in accordance with an embodiment of the present invention an assembly including an obstruction device including a proximal obstruction balloon and a distal obstruction balloon mounted on a shaft, the balloons being inflatable via one or more inflation lumens, and a space-occupying device mounted on a positioning member that extends from a portion of the obstruction device, the space-occupying device being proximal to the proximal obstruction balloon and inflatable via a space-occupying-device inflation lumen.

The positioning member may be straight or not straight (e.g., curved, bent or made of pivoting links and the like). The positioning member may be inflatable. The positioning member may be constructed of a shape memory material.

In accordance with an embodiment of the present invention at least one of the proximal obstruction balloon, the distal obstruction balloon and the space-occupying device may include internal or external anchoring arms.

In accordance with an embodiment of the present invention the positioning member is sized and shaped to position the space-occupying device in a fundus of a patient when the proximal obstruction balloon is placed on a proximal side of a pylorus of the patient and the distal obstruction balloon is placed on a distal side of the pylorus.

There is also provided in accordance with an embodiment of the present invention a method including using the assembly to place the proximal obstruction balloon on the proximal side of a pylorus of a patient and the distal obstruction balloon on a distal side of the pylorus, and using the positioning member to position the space-occupying device in the fundus of the patient.

### BRIEF DESCRIPTION OF THE DRAWINGS

The present invention will be understood and appreciated more fully from the following detailed description taken in conjunction with the drawings in which:

FIGS. 1A and 1B are simplified illustrations of an obstruction assembly, constructed and operative in accordance with an embodiment of the present invention, respectively before and after inflation of a space-occupying device connected to proximal and distal balloons; and

FIGS. 2A-2C are simplified illustrations of a method of using the obstruction assembly, in accordance with an embodiment of the present invention, wherein FIG. 2A illustrates introduction of the assembly through the esophagus to the stomach, FIG. 2B illustrates inflation of the proximal and distal balloons on opposite sides of the pylorus, and FIG. 2C illustrates placement and inflation of the space-occupying device in the fundus.

### DETAILED DESCRIPTION OF EMBODIMENTS

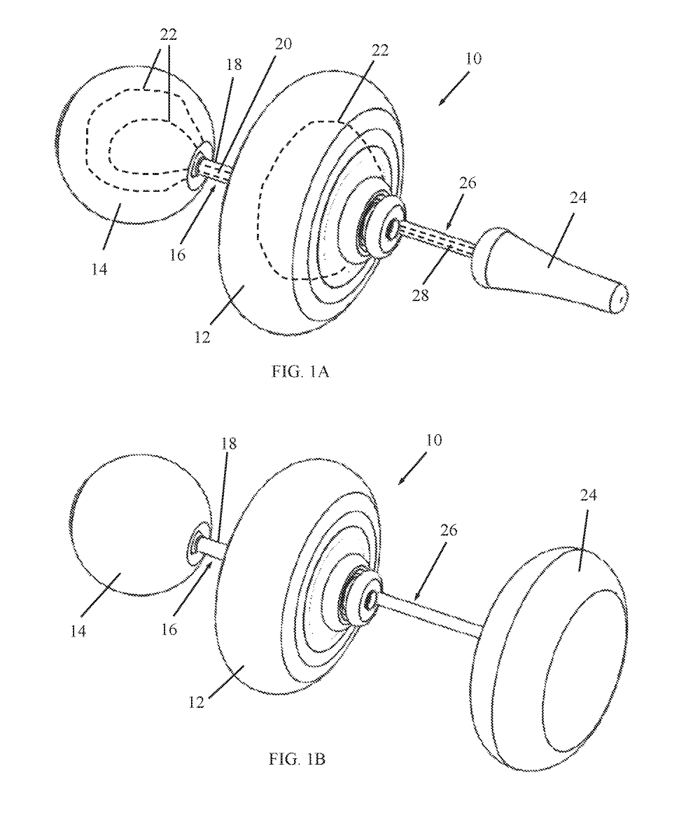

Reference is now made to FIG. 1, which illustrates an obstruction assembly 10, constructed and operative in accordance with an embodiment of the present invention.

Obstruction assembly 10 includes a proximal obstruction balloon 12 and a distal obstruction balloon 14 mounted on a shaft 16. A portion of shaft 16, referred to as neck 18 or neck portion 18, provides a gap between proximal balloon 12 and distal balloon 14. Neck 18 can have different lengths and thicknesses depending on the application; for example, the dimensions of neck 18 are correlated to the usual width of the pylorus muscle.

The proximal obstruction balloon 12 is arranged to fit in the stomach, whereas the distal obstruction balloon 14 is arranged to fit in the duodenum. When inflated, balloons 12 and 14 expand towards the pylorus from opposite sides on the pylorus, thus fixing the plug 10 in place. The balloons may be inflated via one or more inflation lumens 20 formed in shaft 16, which are in fluid communication with a fluid source (not shown), such as air, water or saline, for example. The inflation lumens 20 may also be in fluid communication with a suction source (not shown) for deflation of the balloons; alternatively, one or more lumens dedicated for deflation may be provided.

In accordance with an embodiment of the present invention, proximal obstruction balloon 12 and/or distal obstruction balloon 14 may include a plurality of internal or external anchoring arms 22. In one embodiment, anchoring arms 22 are constructed from folds in balloon 14. In another embodiment, anchoring arms 22 may be arcuate loops of a flexible yet strong material suitable for anchoring against the intestinal walls, such as but not limited to, NITINOL or stainless steel alloy. One purpose of arms 22 is to help anchor the device against the pylorus in the duodenum and/or stomach. Another purpose is to create a non-uniform surface for pushing against tissue. The non-uniform surface may help prevent creating constant pressure, which would have the disadvantageous risk of causing a sore, like a pressure sore, on the tissue.

In accordance with an embodiment of the present invention, a space-occupying device 24 is mounted on a positioning member 26 that extends from a portion of the obstruction device, that is, a portion of proximal obstruction balloon 12, distal obstruction balloon 14 or shaft 16. The space-occupying device 24 is proximal to the proximal obstruction balloon 12 and may be a balloon which is inflatable via a space-occupying-device inflation lumen 28 formed in shaft 16 or in another shaft (not shown). The space-occupying device 24 may be deflated through the inflation lumen or another lumen. The balloons 12 and 14 and the space-occupying device 24 may be deflated simultaneously or one at a time.

The positioning member 26 may be a straight rod, tube or other slender elongate member. Alternatively, positioning member 26 may be not straight (e.g., curved, bent or made of pivoting links and the like). Positioning member 26 may be constructed so as to have a contracted orientation for insertion in the stomach and an expanded or deployed orientation for assuming its final position in the stomach. For example, positioning member 26 may be inflatable so that it expands to its final position in the stomach. As another example, positioning member 26 may be constructed of a shape memory material (such as but not limited to, NITINOL) so that it changes its shape from the contracted orientation to its final deployed orientation in the stomach. The positioning member 26 may be collinear with the shaft 16, or may be offset from the shaft 16.

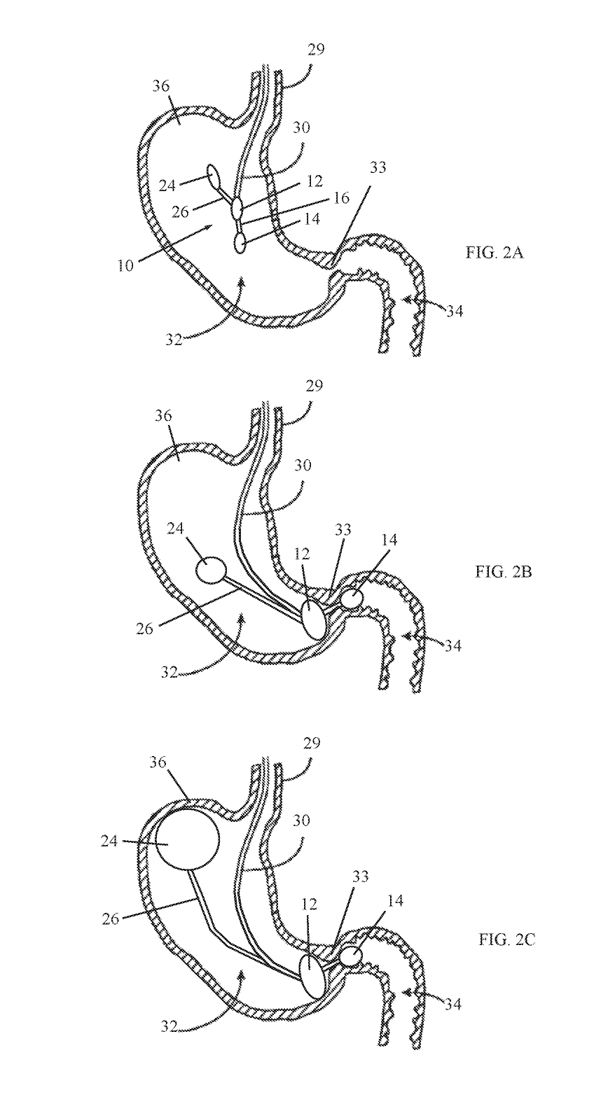

Reference is now made to FIGS. 2A-2C, which illustrate a method of using the obstruction assembly, in accordance with an embodiment of the present invention.

FIG. 2A illustrates introduction of the assembly through the esophagus 29 to the stomach 32 with an endoscopic tool 30. FIG. 2B illustrates inflation of the proximal balloon 12 and the distal balloon 14 on opposite sides of the pylorus 33 (the distal balloon 14 is positioned in the duodenum 34). FIG. 2C illustrates placement and inflation of the space-occupying device 24 in the fundus 36.

Accordingly, the positioning member 26 is sized and shaped to position the space-occupying device 24 in its final deployed orientation in the stomach, such as the fundus of the patient, when the proximal obstruction balloon 12 is placed on the proximal side of the pylorus of the patient and the distal obstruction balloon 14 is placed on the distal side of the pylorus.

What is claimed is:

1. An assembly comprising:
an obstruction device comprising a proximal obstruction balloon and a distal obstruction balloon mounted on a shaft, said balloons being inflatable via one or more inflation lumens; and
a space-occupying device mounted on a positioning member that extends from a portion of said obstruction device, said space-occupying device being proximal to said proximal obstruction balloon and inflatable via a space-occupying-device inflation lumen.

2. The assembly according to claim 1, wherein said positioning member is straight.

3. The assembly according to claim 1, wherein said positioning member is not straight.

4. The assembly according to claim 1, wherein said positioning member is inflatable.

5. The assembly according to claim 1, wherein said positioning member is constructed of a shape memory material.

6. The assembly according to claim 1, wherein at least one of said proximal obstruction balloon, said distal obstruction balloon and said space-occupying device comprises internal or external anchoring arms.

7. The assembly according to claim 1, wherein said positioning member is sized and shaped to position said space-occupying device in a fundus of a patient when said proximal obstruction balloon is placed on a proximal side of a pylorus of the patient and said distal obstruction balloon is placed on a distal side of the pylorus.

8. A method comprising using said assembly of claim 1 to place said proximal obstruction balloon on a proximal side of a pylorus of a patient and said distal obstruction balloon on a distal side of the pylorus, and using said positioning member to position said space-occupying device in a fundus of the patient.
